(19) United States
(12) Patent Application Publication
(10) Pub. No.: US 2015/0005846 A1
(43) Pub. Date: Jan. 1, 2015

(54) ELECTRICAL DETERMINATION OF THE LOCAL PHYSIOLOGICAL ENVIRONMENT IN SPINAL CORD STIMULATION

(71) Applicant: BOSTON SCIENTIFIC NEUROMODULATION CORPORATION, Valencia, CA (US)

(72) Inventors: Emarit A.S. Ranu, Fort Collins, CO (US); Changfang Zhu, Valencia, CA (US); Dongchul Lee, Agua Dulce, CA (US); Bradley L. Hershey, Valencia, CA (US)

(21) Appl. No.: 14/312,426

(22) Filed: Jun. 23, 2014

Related U.S. Application Data
(60) Provisional application No. 61/841,855, filed on Jul. 1, 2013.

(51) Int. Cl. A61N 1/36; A61N 1/372

(52) U.S. Cl. CPC A61N 1/36128; A61N 1/37235
USPC 607/59; 607/62

(57) ABSTRACT

A medical system configured for performing a medical function in a patient comprises a medical lead configured for being implanted adjacent a tissue region of the patient, and an electrode configured for being implanted adjacent the tissue region. The medical system further comprises analog output circuitry configured for delivering one or more electrical signals having a plurality of different sinusoidal frequency components to the tissue region via the electrode, monitoring circuitry configured for measuring a plurality of different frequency-dependent or other basis function electrical parameter values in response to the delivery of the one or more electrical signals to the tissue region, and at least one controller/processor configured for analyzing the different electrical parameter values, and performing a function based on the different analyzed electrical parameter values.

### RELATED APPLICATION DATA

The present application claims the benefit under 35 U.S.C. §119 to U.S. Provisional Patent Application No. 61/841,855, filed Jul. 1, 2013. The foregoing application is hereby incorporated by reference into the present application in its entirety.

### FIELD OF THE INVENTION

The present invention relates to implantable medical systems, and more particularly, to apparatus and methods for the local physiological environment of tissue in which electrical stimulation leads are implanted.

### BACKGROUND OF THE INVENTION

Implantable neurostimulation systems have proven therapeutic in a wide variety of diseases and disorders. Pacemakers and Implantable Cardiac Defibrillators (ICDs) have proven highly effective in the treatment of a number of cardiac conditions (e.g., arrhythmias). Spinal Cord Stimulation (SCS) systems have long been accepted as a therapeutic modality for the treatment of chronic pain syndromes, and the application of tissue stimulation has begun to expand to additional applications such as angina pectoralis and incontinence. Deep Brain Stimulation (DBS) has also been applied therapeutically for well over a decade for the treatment of refractory chronic pain syndromes, and DBS has also recently been applied in additional areas such as movement disorders and epilepsy. Further, Functional Electrical Stimulation (FES) systems have been applied to restore some functionality to paralyzed extremities in spinal cord injury patients. Furthermore, in recent investigations Peripheral Nerve Stimulation (PNS) systems have demonstrated efficacy in the treatment of chronic pain syndromes and incontinence, and a number of additional applications are currently under investigation. Occipital Nerve Stimulation (ONS), in which leads are implanted in the tissue over the occipital nerves, has shown promise as a treatment for various headaches, including migraine headaches, cluster headaches, and cervicogenic headaches.

These implantable neurostimulation systems typically include one or more electrode carrying stimulation leads, which are implanted at the desired stimulation site, and a neurostimulator (e.g., an implantable pulse generator (IPG)) implanted remotely from the stimulation site, but coupled either directly to the stimulation lead(s) or indirectly to the stimulation lead(s) via a lead extension. Thus, electrical pulses can be delivered from the neurostimulator to the stimulation leads to stimulate the tissue and provide the desired efficacious therapy to the patient. The neurostimulation system may further comprise a handheld patient programmer in the form of a remote control (RC) to remotely instruct the neurostimulator to generate electrical stimulation pulses in accordance with selected stimulation parameters. A typical stimulation parameter set may include the electrodes that are acting as anodes or cathodes, as well as the amplitude, duration, and rate of the stimulation pulses.

Thus, the RC can be used to instruct the neurostimulator to generate electrical stimulation pulses in accordance with the selected stimulation parameters. Typically, the stimulation parameters programmed into the neurostimulator can be adjusted by manipulating controls on the RC to modify the electrical stimulation provided by the neurostimulator system to the patient. Thus, in accordance with the stimulation parameters programmed by the RC, electrical pulses can be delivered from the neurostimulator to the stimulation electrode(s) to stimulate or activate a volume of tissue in accordance with a set of stimulation parameters and provide the desired efficacious therapy to the patient. The best stimulus parameter set will typically be one that delivers stimulation energy to the volume of tissue that must be stimulated in order to provide the therapeutic benefit (e.g., treatment of pain), while minimizing the volume of non-target tissue that is stimulated.

The IPG may be programmed by a clinician, for example, by using a clinician's programmer (CP), which typically includes a general purpose computer, such as a laptop, with a programming software package installed thereon. Typically, the RC can only control the neurostimulator in a limited manner (e.g., by only selecting a program or adjusting the pulse amplitude or pulse width), whereas the CP can be used to control all of the stimulation parameters, including which electrodes are cathodes or anodes.

In the context of an SCS procedure, one or more stimulation leads are introduced through the patient's back into the epidural space, such that the electrodes carried by the leads are arranged in a desired pattern and spacing to create an electrode array. One type of commercially available stimulation leads is a percutaneous lead, which comprises a cylindrical body with ring electrodes, and can be introduced into contact with the affected spinal tissue through a Touhy-like needle, which passes through the skin, between the desired vertebrae, and into the epidural space above the dura layer. After proper placement of the stimulation leads at the target area of the spinal cord, the leads are anchored in place at an exit site to prevent movement of the stimulation leads.

To facilitate the location of the neurostimulator away from the exit point of the stimulation leads, lead extensions are sometimes used. The stimulation leads, or the lead extensions, are then connected to the IPG, which can then be operated to generate electrical pulses that are delivered, through the electrodes, to the targeted tissue, and in particular, the dorsal column and dorsal root fibers within the spinal cord. Intra-operatively (i.e., during the surgical procedure), the neurostimulator may be operated to test the effect of stimulation and adjust the parameters of the stimulation for optimal pain relief. A computer program, such as Bionic Navigator®, available from Boston Scientific Neuromodulation Corporation, can be incorporated in a clinician's programmer (CP) (briefly discussed above) to facilitate selection of the stimulation parameters. Any incisions are then closed to fully implant the system. Post-operatively (i.e., after the surgical procedure has been completed), a clinician can adjust the stimulation parameters using the computerized programming system to re-optimize the therapy.

After implantation of the stimulation leads, it may be desirable to electrically monitor the physiological environment in which the stimulation leads have been implanted in order to perform any one of various functions.

For example, the efficacy of SCS is related to the ability to stimulate the spinal cord tissue that inervates the region of pain experienced by the patient. Thus, the working clinical paradigm is that achievement of an effective result from SCS depends on the neurostimulation lead or leads being placed in a location (both longitudinal, lateral, and depth) relative to the spinal tissue, such that the electrical stimulation will treat the region of pain (i.e., the target of treatment). If a lead is not correctly positioned, it is possible that the patient will receive little or no benefit from an implanted SCS system. Thus, correct lead placement can mean the difference between effective and ineffective pain therapy, and as such, precise positioning of the leads proximal to the targets of stimulation is critical to the success of the therapy.

For example, multi-lead configurations, which enable more programming options for optimizing therapy, have been increasingly used in SCS applications. The use of multiple leads that are grouped together in close proximity to each other at one general region of the patient (e.g., side-by-side parallel leads along the spinal cord of the patient), increases the stimulation area and penetration depth (therefore coverage), as well as enables more combinations of anodic and cathodic electrodes for stimulation, such as transverse multipolar (bipolar, tripolar, or quadra-polar) stimulation, in addition to any longitudinal single lead configuration. Furthermore, with these lead configurations, current can be manipulated between leads medio-laterally to create the desired stimulation field. The resulting stimulation field is highly dependent on the relative position of the electrodes selected for stimulation.

Although the stimulation lead(s) may initially be correctly positioned relative to each other or relative to the stimulation target(s), the stimulation lead(s) are at risk of migration relative to each other and/or relative to the stimulation target(s). The stimulation lead(s) may migrate both acutely (e.g., during posture change or during activity/exercise) or chronically. In the context of SCS, the stimulation lead(s) may potentially migrate in three dimensions: rostro-caudally (along the axis of the spinal cord), medio-laterally (lateral to the spinal cord), and dorsal-ventrally (depth of the lead relative to the spinal cord). Notably, because the thickness of the cerebral spinal fluid (CSF) between the stimulation lead(s) and the spinal cord vary along the length spinal cord, migration of the stimulation lead(s) in the rostro-caudal direction may necessarily in the lead(s) being subjected to a different volume of CSF. Once the leads(s) migrate from their original position, a corrective action, such as surgical repositioning or electronic reprogramming of the stimulation leads may need to be performed relocate the stimulation to the targeted tissue region. Further details discussing the detection of lead migration by measuring electrical parameters, such as impedance, field potential, and evoked action potentials, are provided in U.S. Pat. Nos. 7,684,869, 7,853,330, and 8,401,665, which are expressly incorporated herein by reference.

As another example of a reason for electrically monitoring the physiological environment of the stimulation leads is that the coupling efficiency between the active electrodes and the targeted tissue region may change (either increase or decrease) as a result of inherent changes in the tissue characteristics typically caused by the tissue encapsulation process, which eventually surrounds the stimulation lead(s) with fibrous collagenous tissue (i.e., scar tissue) in an attempt to isolate the foreign materials of the stimulation lead(s). If the coupling efficiency decreases as a result of the tissue encapsulation process (or other processes), the intensity of the stimulation may be too low to provide effective therapy, whereas if the coupling efficiency increases as a result of the tissue encapsulation process (or other processes), the intensity of the stimulation may be too high and may overstimulate the targeted tissue region, inadvertently stimulate non-targeted tissue, and/or waste energy. Thus, knowledge of the coupling efficiency between the electrodes and the target tissue will allow the intensity of the stimulation to be adjusted to provide for a safe and efficacious level of therapy. In one preferred embodiment, the impedance between the electrodes and the target tissue is measured to determine the coupling efficiency, such that the amplitude of the stimulation can be automatically adjusted, as described in U.S. Pat. No. 7,742,823, which is expressly incorporated herein by reference.

In addition to tracking the coupling efficiency between the electrodes and the target tissue, it may be desirable to provide insight into the state of the encapsulation process (e.g., if the scar tissue has matured, is developing, is nascent, or even absent, etc.), thereby providing an indication of the stability of the stimulation lead(s). For example, if the encapsulation process is in the early stages, the activity of the patient may be limited so that the encapsulation process is not disrupted. In contrast, if the encapsulation process is complete, the stimulation lead(s) may be stabilized, and thus, no physical limitations may be placed on the patient.

As still another example of a reason for electrically monitoring the physiological environment of the stimulation leads is that it may be desirable to track the physical activity (e.g., activity level or body manipulations) of the patient that has received the implantable neurostimulation system, which provides an indication of the efficacy of the therapy provided by the stimulation system; that is, the more efficacious the therapy, the more diurnally active the patient will be. Thus, knowledge of the physical activity of the patient over a period of time in which therapeutic stimulation is applied to the patient may be used by a physician or clinician to prescribe pharmaceuticals, reprogram or upgrade the IPG, or implement or modify other therapeutic regimens (such as physical or occupational therapy). Knowledge of the physical activity of the patient may also be used to adapt the therapy provided by the stimulation system in real time, so that the stimulation is consistently provided to the patient at an efficacious and/or comfortable level. Further details discussing the tracking of the physical activity of a patient are provided in U.S. patent application Ser. No. 12/024,947, entitled “Neurostimulation System and Method for Measuring Patient Activity,” which is expressly incorporated herein by reference.

There remains a need to provide improved techniques for characterizing the tissue surrounding a medical lead.

### SUMMARY OF THE INVENTION

In accordance with the present inventions, a medical system configured for performing a medical function in a patient is provided. The medical system comprises a medical lead configured for being implanted adjacent a tissue region of the patient, and an electrode (which may be carried by the medical lead) configured for being implanted adjacent the tissue region. The medical system further comprises analog output circuitry configured for delivering one or more electrical signals having a plurality of different sinusoidal frequency components to the tissue region via the electrode. The medical system further comprises monitoring circuitry configured for measuring a plurality of different frequency-dependent electrical parameter values (e.g., impedance values, which may be one or both of a resistance or reactance) in response to the delivery of the electrical signal(s) to the tissue region.

The medical system further comprises at least one controller/processor configured for analyzing the different electrical parameter values, and performing a function (e.g., determining migration of the lead, determining a state of an encapsulation process with respect to the medical lead, determining a posture and/or physical activity of the patient) based on the different analyzed electrical parameter values.

In one embodiment, the electrical signal(s) comprises at least one monopolar electrical signal and at least one multipole electrical signal, the electrical parameter values comprise a plurality of monopolar impedance values and a plurality of multipolar impedance values, and the controller/processor is configured for analyzing the electrical parameter values by computing differences between the monopolar impedance values and the multipolar impedance values. In another embodiment, the medical system further comprises memory storing at least one reference value, in which case, the controller/processor may be configured for analyzing the electrical parameter values by comparing the electrical parameter values to the reference value(s), and performing the function based on the comparison.

If a single electrical signal (e.g., an electrical pulse train) is delivered to the tissue via the electrode, the monitoring circuitry may include a frequency spectrum analyzer (implemented via hardware or software) configured for measuring the electrical parameter values at frequencies corresponding to the different sinusoidal frequency components. If multiple electrical signals having different frequency component profiles (e.g., a plurality of electrical pulse trains having different pulse rates and/or different pulse widths or a plurality of sinusoidal signals having different frequencies) are delivered to the tissue via the electrode, the monitoring circuitry may be configured for measuring the electrical parameter values respectively in response to the delivery of the different electrical signals to the tissue region. The function performed by the controller/processor may further comprise performing a corrective action, such as reprogramming at least one electrode on the medical lead and/or providing a notification message or warning to the user.

In one embodiment, the medical system further comprises an implantable casing containing the analog output circuitry, monitoring circuitry, and the controller/processor. In another embodiment, the medical system further comprises an external control device containing the controller/processor(s).

Other and further aspects and features of the invention will be evident from reading the following detailed description of the preferred embodiments, which are intended to illustrate, not limit, the invention.

### BRIEF DESCRIPTION OF THE DRAWINGS

The drawings illustrate the design and utility of preferred embodiments of the present invention, in which similar elements are referred to by common reference numerals. In order to better appreciate how the above-recited and other advantages and objects of the present inventions are obtained, a more particular description of the present inventions briefly described above will be rendered by reference to specific embodiments thereof, which are illustrated in the accompanying drawings. Understanding that these drawings depict only typical embodiments of the invention and are not therefore to be considered limiting of its scope, the invention will be described and explained with additional specificity and detail through the use of the accompanying drawings in which:

FIG. 1 is plan view of one embodiment of a spinal cord stimulation (SCS) system arranged in accordance with the present inventions;

FIG. 2 is a plan view of an implantable pulse generator (IPG) and two neurostimulation leads used in the SCS system of FIG. 1;

FIG. 3 is a plan view of the SCS system of FIG. 1 in use with a patient;

FIGS. 4a-4h are diagrams illustrating different electrical signals in the time-domain and the frequency domain, which can be delivered by the SCS system 10 to measure impedance values;

FIG. 5 is a block diagram of the internal components of the IPG of FIG. 1;

FIG. 6 is a plan view of a remote control that can be used in the SCS system of FIG. 1; and

FIG. 7 is a block diagram of the internal componentry of the remote control of FIG. 6.

### DETAILED DESCRIPTION OF THE EMBODIMENTS

The description that follows relates to a spinal cord stimulation (SCS) system. However, it is to be understood that while the invention lends itself well to applications in SCS, the invention, in its broadest aspects, may not be so limited. Rather, the invention may be used with any type of implantable electrical circuitry used to stimulate tissue. For example, the present invention may be used as part of a multi-lead system such as a pacemaker, a defibrillator, a cochlear stimulator, a retinal stimulator, a stimulator configured to produce coordinated limb movement, a cortical stimulator, a deep brain stimulator, peripheral nerve stimulator, microstimulator, or in any other neural stimulator configured to treat urinary incontinence, sleep apnea, shoulder sublaxation, headache, etc.

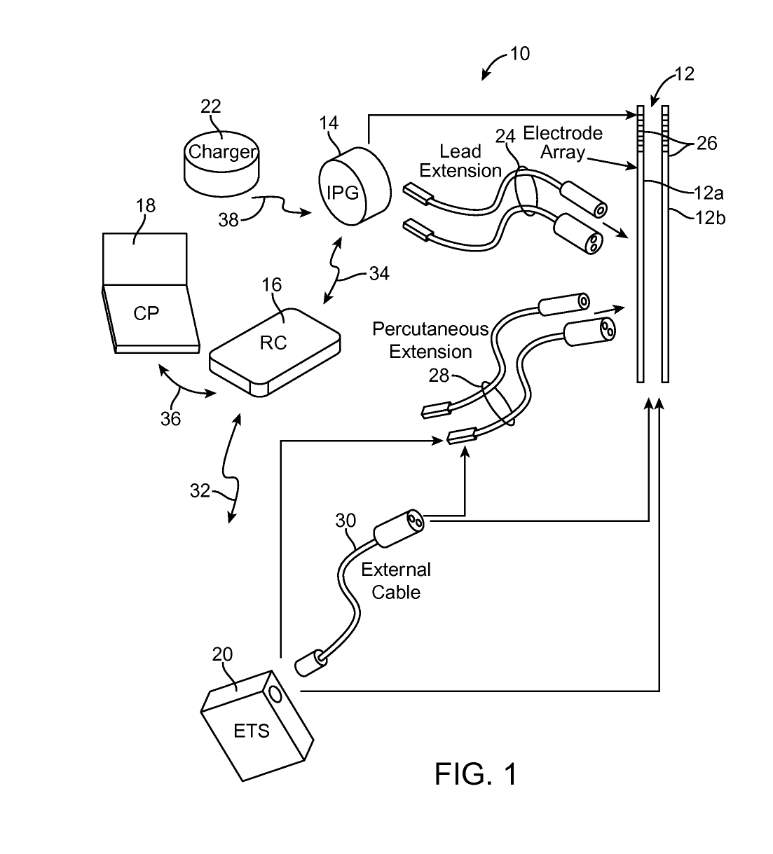

Turning first to FIG. 1, an exemplary SCS system 10 generally comprises a plurality of neurostimulation leads 12 (in this case, two percutaneous leads 12a and 12b), an implantable pulse generator (IPG) 14, an external remote control (RC) 16, a Clinician's Programmer (CP) 18, an External Trial Stimulator (ETS) 20, and an external charger 22.

The IPG 14 is physically connected via two lead extensions 24 to the neurostimulation leads 12, which carry a plurality of electrodes 26 arranged in an array. As will also be described in further detail below, the IPG 14 includes pulse generation circuitry that delivers electrical stimulation energy in the form of a pulsed electrical waveform (i.e., a temporal series of electrical pulses) to the electrode array 26 in accordance with a set of stimulation parameters. The IPG 14 and neurostimulation leads 12 can be provided as an implantable neurostimulation kit, along with, e.g., a hollow needle, a stylet, a tunneling tool, and a tunneling straw. Further details discussing implantable kits are disclosed in U.S. Application Ser. No. 61/030,506, entitled “Temporary Neurostimulation Lead Identification Device,” which is expressly incorporated herein by reference.

The ETS 20 may also be physically connected via percutaneous lead extensions 28 or external cable 30 to the neurostimulation lead 12. The ETS 20, which has similar pulse generation circuitry as the IPG 14, also delivers electrical stimulation energy in the form of a pulse electrical waveform to the electrode array 26 in accordance with a set of stimulation parameters. The major difference between the ETS 20 and the IPG 14 is that the ETS 20 is a non-implantable device that is used on a trial basis after the neurostimulation lead 12 has been implanted and prior to implantation of the IPG 14, to test the responsiveness of the stimulation that is to be provided. Thus, any functions described herein with respect to the IPG 14 can likewise be performed with respect to the ETS 20.

The RC 16 may be used to telemetrically control the ETS 20 via a bi-directional RF communications link 32. Once the IPG 14 and stimulation leads 12 are implanted, the RC 16 may be used to telemetrically control the IPG 14 via a bi-directional RF communications link 34. Such control allows the IPG 14 to be turned on or off and to be programmed with different stimulation programs after implantation. Once the IPG 14 has been programmed, and its power source has been charged or otherwise replenished, the IPG 14 may function as programmed without the RC 16 being present.

The CP 18 provides clinician detailed stimulation parameters for programming the IPG 14 and ETS 20 in the operating room and in follow-up sessions. The CP 18 may perform this function by indirectly communicating with the IPG 14 or ETS 20, through the RC 16, via an IR communications link 36. Alternatively, the CP 18 may directly communicate with the IPG 14 or ETS 20 via an RF communications link (not shown).

The external charger 22 is a portable device used to transcutaneously charge the IPG 14 via an inductive link 38. Once the IPG 14 has been programmed, and its power source has been charged by the external charger 22 or otherwise replenished, the IPG 14 may function as programmed without the RC 16 or CP 18 being present.

For purposes of brevity, the details of the CP 18, ETS 20, and external charger 22 will not be described herein. Details of exemplary embodiments of these components are disclosed in U.S. Pat. No. 6,895,280, which is expressly incorporated herein by reference.

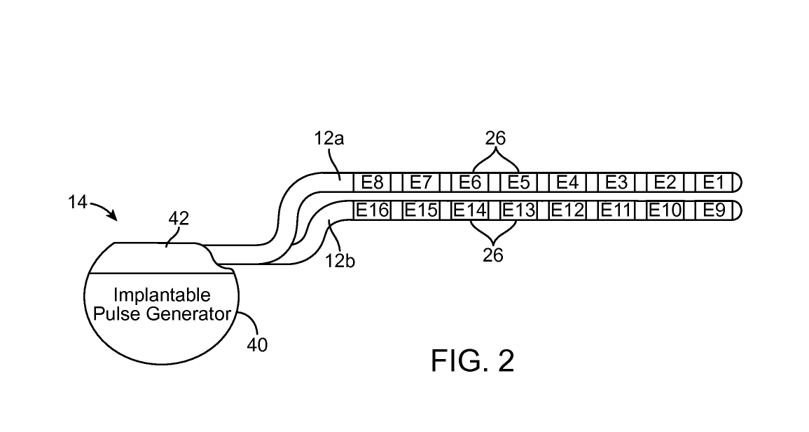

Referring now to FIG. 2, the external features of the neurostimulation leads 12a, 12b and the IPG 14 will be briefly described. Each of the neurostimulation leads 12 has eight electrodes 26 (respectively labeled E1-E8 for the lead 12a and E9-E16 for the lead 12b). The actual number and shape of leads and electrodes will, of course, vary according to the intended application. Further details describing the construction and method of manufacturing percutaneous stimulation leads are disclosed in U.S. patent application Ser. No. 11/689,918, entitled “Lead Assembly and Method of Making Same,” and U.S. patent application Ser. No. 11/565,547, entitled “Cylindrical Multi-Contact Electrode Lead for Neural Stimulation and Method of Making Same,” the disclosures of which are expressly incorporated herein by reference.

The IPG 14 comprises an outer case 40 for housing the electronic and other components (described in further detail below). The outer case 40 is composed of an electrically conductive, biocompatible material, such as titanium, and forms a hermetically sealed compartment wherein the internal electronics are protected from the body tissue and fluids. In some cases, the outer case 40 may serve as an electrode. The IPG 14 further comprises a connector 42 to which the proximal ends of the neurostimulation leads 12 mate in a manner that electrically couples the electrodes 26 to the internal electronics (described in further detail below) within the outer case 40. To this end, the connector 42 includes two ports (not shown) for receiving the proximal ends of the percutaneous leads 12. In the case where the lead extensions 24 are used, the ports may instead receive the proximal ends of such lead extensions 24.

As briefly discussed above, the IPG 14 includes circuitry that provides electrical stimulation energy to the electrodes 26 in accordance with a set of parameters. Such stimulation parameters may comprise electrode combinations, which define the electrodes that are activated as anodes (positive), cathodes (negative), and turned off (zero), percentage of stimulation energy assigned to each electrode (fractionalized electrode configurations), and electrical pulse parameters, which define the pulse amplitude (measured in milliamps or volts depending on whether the IPG 14 supplies constant current or constant voltage to the electrode array 26), pulse width (measured in microseconds), pulse rate (measured in pulses per second), and burst rate (measured as the stimulation on duration X and stimulation off duration Y). As will be described in further detail below, the IPG 14 also includes circuitry that provides electrical signals, and measured electrical impedance in response to the electrical signals.

With respect to the pulse patterns provided during operation of the SCS system 10, electrodes that are selected to transmit or receive electrical energy are referred to herein as “activated,” while electrodes that are not selected to transmit or receive electrical energy are referred to herein as “non-activated.” Electrical energy delivery will occur between two (or more) electrodes, one of which may be the IPG case 40, so that the electrical current has a path from the energy source contained within the IPG case 40 to the tissue and a sink path from the tissue to the energy source contained within the case. Electrical energy may be transmitted to the tissue in a monopolar or multipolar (e.g., bipolar, tripolar, etc.) fashion.

Monopolar delivery occurs when a selected one or more of the lead electrodes 26 is activated along with the case 40 of the IPG 14, so that electrical energy is transmitted between the selected electrode 26 and case 40. Monopolar delivery may also occur when one or more of the lead electrodes 26 are activated along with a large group of lead electrodes located remotely from the one or more lead electrodes 26 so as to create a monopolar effect; that is, electrical energy is conveyed from the one or more lead electrodes 26 in a relatively isotropic manner. Bipolar delivery occurs when two of the lead electrodes 26 are activated as anode and cathode, so that electrical energy is transmitted between the selected electrodes 26. Tripolar delivery occurs when three of the lead electrodes 26 are activated, two as anodes and the remaining one as a cathode, or two as cathodes and the remaining one as an anode.

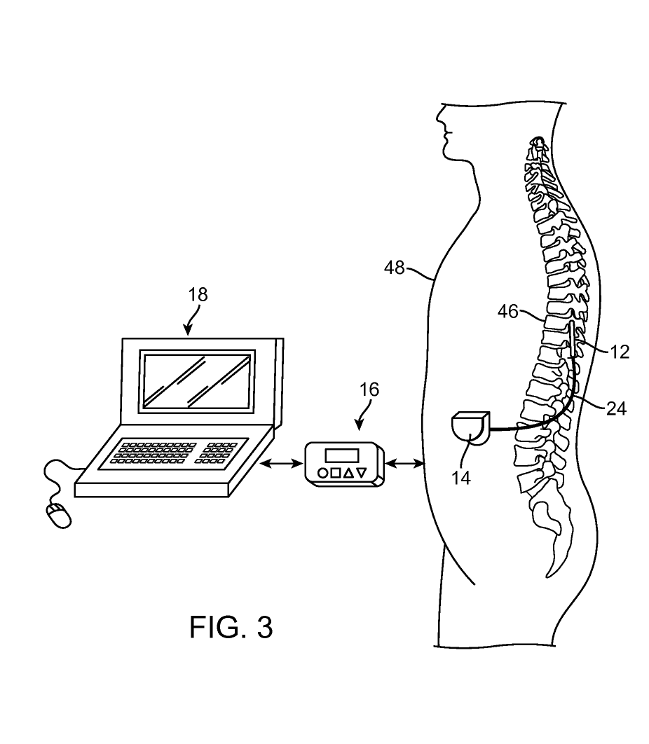

Referring to FIG. 3, the neurostimulation leads 12 are implanted at an initial position within the spinal column 46 of a patient 48. The preferred placement of the neurostimulation leads 12 is adjacent, i.e., resting near, or upon the dura, adjacent to the spinal cord area to be stimulated. Due to the lack of space near the location where the neurostimulation leads 12 exit the spinal column 46, the IPG 14 is generally implanted in a surgically-made pocket either in the abdomen or above the buttocks. The IPG 14 may, of course, also be implanted in other locations of the patient's body. The lead extensions 24 facilitate locating the IPG 14 away from the exit point of the neurostimulation leads 12. As there shown, the CP 18 communicates with the IPG 14 via the RC 16. After implantation, the IPG 14 can be operated to generate a volume of activation relative to the target tissue to be treated, thereby providing the therapeutic stimulation under control of the patient.

As previously mentioned in the background of the invention, the tissue environment in which the neurostimulation leads 12 may change due to lead migration (either relative to each other or relative to a point in reference point in the tissue), tissue encapsulation, posture changes, patient activity, etc. Significantly, the SCS system 10 takes advantage of the fact that biological materials (e.g., tissue) exhibit frequency dependent characteristics when measured via electrical means. In other words, biological materials can have an electrical impedance with both a resistive (real) and reactive (imaginary) component that, independently or together, are a function of frequency. By characterizing this impedance along each of the electrodes 26 and at various frequencies, the properties of the tissue in which the stimulation leads 12 are implanted can be more accurately determined in contrast to prior art techniques, which take only one measurement at each electrode without regard to the frequency response of the tissue. That is, whereas the prior art techniques analyze the electrical signals in one frequency dimension, the techniques performed by the SCS system 10 analyze the electrical signals in multiple frequency dimensions to create frequency vectors that may better characterize the tissue.

To this end, the SCS system 10 is configured for delivering at least one electrical signal having a plurality of different sinusoidal frequency components to the tissue region (in this case, the targeted spinal cord tissue region) of the patient via each electrode 26. That is, the SCS system 10 will deliver electrical signal(s) to a first one of the electrodes 26, then deliver electrical signal(s) to a second one of the electrodes 26, then deliver electrical signal(s) to a third one of the electrodes 26, etc. Alternatively, the SCS system 10 will only deliver electrical signal(s) to each of a subset of electrodes 26 (i.e., less than all of the available electrodes 26 including only one of the electrodes 26). As another alternative, the SCS system 10 can deliver electrical signal(s) to an electrode or electrodes (not shown) not carried by the stimulation leads 12. The object is to characterize the tissue adjacent the stimulation leads 12. The electrical signals delivered by the SCS system 10 to the tissue are preferably sub-threshold; that is, they have an intensity that is low enough to avoid eliciting a physiological response, but high enough to obtain an accurate measurement. Alternatively, as mentioned in further detail below, the electrical signals may be super-threshold, and may even include the therapeutic electrical pulse trains.

As is well known, an electrical signal can be represented as a sum of its frequency components by calculating its Fourier series coefficients.

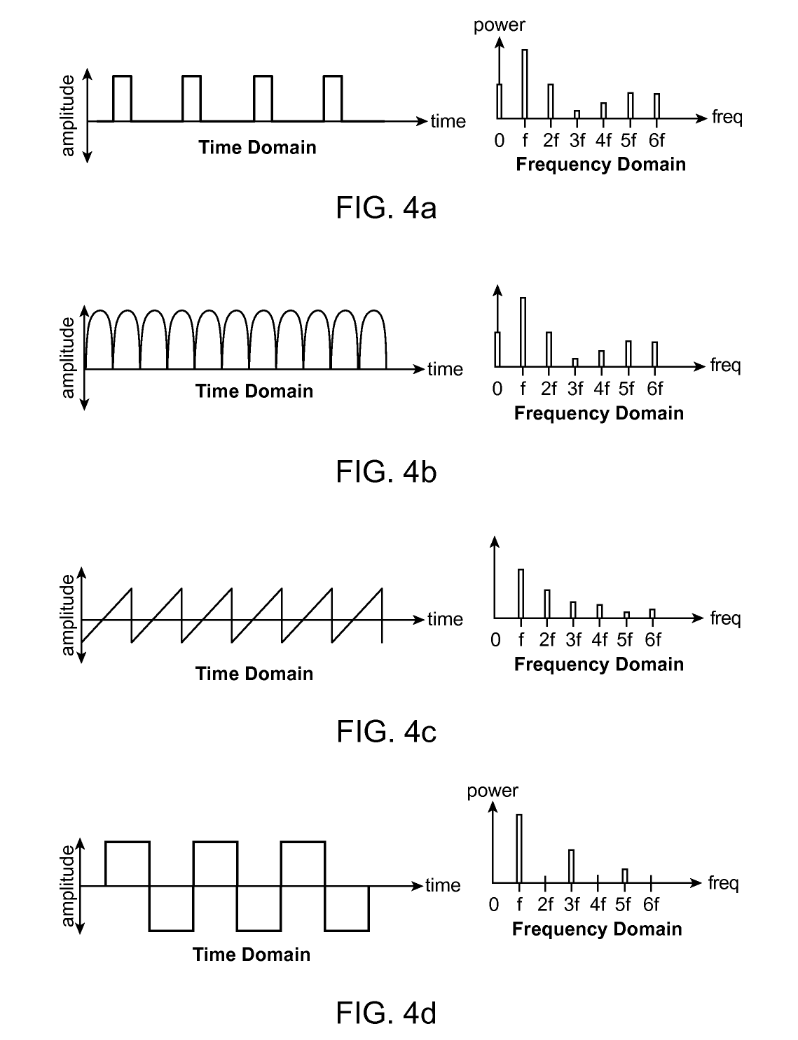

A periodic electrical signal will typically have frequency components represented by discrete energy spectrum components. For example, a rectangular electrical pulse train will have discrete energy components at zero and at both even and odd harmonics of the pulse rate frequency f (i.e., the fundamental frequency), as illustrated in FIG. 4a. The pulse rate of the electrical pulse train can be selected to influence what frequencies are applied to the tissue, and the pulse width of the electrical pulse train can be selected to influence what fraction of energy is applied to the tissue at each respective frequency. Similarly, a rectified electrical signal will have discrete energy components at a 0, as well as at both even and odd harmonics of the pulse rate frequency f, as illustrated in FIG. 4b. A sawtooth electrical pulse train will have discrete energy components at both even and odd harmonics of the pulse rate frequency f, as illustrated in FIG. 4b. In contrast, a square electrical pulse train will have discrete energy components only at odd harmonics of the pulse rate frequency f, as illustrated in FIG. 4d. Similarly, a triangular electrical pulse train will have discrete energy components at only the odd harmonics of the pulse rate frequency f, as illustrated in FIG. 4e. A pure sinusoidal electrical signal will have a single energy component at the frequency of the sinusoidal frequency, as illustrated in FIG. 4f.

In contrast to periodic electrical signals, non-periodic electrical signals will typically have frequency components represented by continuous spectrum components. For example, an electrical signal with a single pulse will have continuous energy components in the shape of a continuous waveform having nulls at odd harmonics of a frequency equal to the inverse of the pulse width 6, as illustrated in FIG. 4g. An impulse electrical signal (spike with infinitesimal width) will have uniform energy components across an infinite range of frequencies, as illustrated in FIG. 4h.

In response to the delivery of the electrical signal(s) to the tissue region via a particular electrode 26, the SCS system 10 measures a plurality of different frequency-dependent electrical parameter values at that electrode 26. In the preferred embodiment, these electrical parameter values are impedance values, although in an alternative embodiment, the electrical parameter values can be, e.g., field potential values. Because both the resistive (real) and reactive (imaginary) components of biological tissue impedance vary with frequency of an applied electrical signal, either of the resistive and reactive components of the relevant electrical parameter or the entire complex magnitude of the relevant electrical parameter can be measured. In general, magnitudes of the real and reactive components, and thus the total complex magnitude, of the tissue impedance inversely varies with the frequency of the applied electrical signal. The tissue impedance measurements taken at each of the electrodes 26 may be, e.g., monopolar (i.e., impedance measured between the respective electrode 26 and the case electrode and/or multipolar (i.e., impedance measured between the respective electrode 26 and another one of the lead electrodes 26). The tissue impedance values may be measured in the time-domain or the frequency domain or via any other signal decomposition methodology, such as wavelets, or using any of a variety of basis functions.

In one embodiment, multiple electrical signals having different frequency component profiles may be delivered to each electrode. For example, a series of sinusoidal electrical signals having different frequencies can be delivered to each electrode. In response to the delivery of each sinusoidal signal, the impedance (whether real, imaginary, or complex) can be measured at the respective electrode in the time-domain. Since the sinusoidal signals differ from each other in frequency, it is expected that the impedances measured in response to the electrical pulse trains will have different values. As will be described in further detail below, it is this variance in impedance that allows the tissue surrounding each electrode to be more accurately characterized. As another example, a series of electrical pulse trains having different pulse rates and/or pulse widths can be delivered to each electrode. In response to the delivery of each electrical pulse train, the impedance (whether real, imaginary, or complex) can be measured in the time-domain. Since the electrical pulse trains differ from each other in pulse rate and/or pulse width, it is expected that the impedance values measured in response to the electrical pulse trains will vary from each other.

In another embodiment, a single electrical signal (e.g., an electrical pulse train) may be delivered to each electrode. In this case, in response to the delivery of each electrical pulse train, the impedance (whether real, imaginary, or complex) can be measured at the respective electrode in the frequency-domain. For example, the impedance can be measured at the fundamental frequency (i.e., the pulse rate), and the first five multiples of the fundamental frequency (i.e., 2f, 3f, . . . 6f). It is expected that the impedances measured in response to the electrical pulse train at these frequencies will have different values, with the impedance measured at the fundamental frequency having the largest value. Like the case with the multiple electrical signals that are measured in the time domain, the variance in the impedance values measured in the frequency domain in response to the delivery of the single electrical pulse train, allows the tissue surrounding each electrode to be more accurately characterized.

Once the impedance values, whether measured in the time-domain or the frequency domain, are acquired at each of the electrodes 26, the SCS system 10 is configured for analyzing the different electrical parameter values at each of the electrodes 26, and performing a function based on the different analyzed electrical parameter values. Significantly, because there are multiple impedance values associated with each electrode 26, a vector having a number of dimensions equal to the number of impedance values can be plotted in the frequency space. If only the total magnitude of the impedance is taken into account, the number of impedance values will equal the number of frequencies at which the impedance is measured. If the resistive and reactive components of the impedance are taken into account, the number of impedance vectors will be twice the number of frequencies at which the impedance is analyzed. If the total magnitude of the impedance, as well as both resistive and reactive components of the impedance, are taken into account, the number of impedance vectors will be three times the number of frequencies at which the impedance is analyzed. If both monopolar and multipolar impedance measurements are acquired, the monopolar and multipole impedance measurements and/or a function thereof (e.g., the difference) may provide additional dimensions that can be plotted in the frequency space.

The manner in which the electrical parameter values are analyzed may depend on the function performed by the SCS system 10.

For example, if the function to be performed by the SCS system 10 is determining a migration of one or both of the stimulation leads 12, the analysis may involve comparing the impedance values acquired at the respective frequencies for each electrode 26 to impedance values previously acquired (e.g., at the time of implantation) at the respective frequencies for the same electrode 26, and making a determination of whether and how much the stimulation lead 12 has migrated based on the comparison.

In one particular example, at implantation, the impedance values for each electrode 26 can be acquired to create a reference impedance profile for all of the electrodes 26 that is then stored in the SCS system 10. Subsequently, the impedance values for each electrode 26 can be acquired to create a current impedance profile for all of the electrodes 26. Both the reference impedance profile and the current impedance profile can be plotted, with the x-axis represented by the electrode designation, and the remaining axes (the number of which depend on the number of frequencies, or other basis functions, at which the impedance values were measured) represented by the impedance values for the designated electrode. Because the tissue characteristics will vary along the length of the stimulation lead 12, a shift in the plots will indicate whether and the extent to which the stimulation lead 12 has migrated along its axis. Once the extent of lead migration is determined, the SCS system 10 can perform a corrective action, such as programming the electrodes 26 with new stimulation parameters (e.g., new fractionalized current values for the electrodes) in order to restore the original therapy and/or providing a notification message or warning to patient that the one of the stimulation leads 12 has migrated.

As another example, if the function performed by the SCS system 10 includes determining a state of the encapsulation process with respect to one or both of the stimulation leads 12, the analysis may involve comparing the impedance values acquired at the respective frequencies for each electrode 26 to known impedance values at those frequencies as a function of the buildup of fibrous collagen (scar tissue), and making a determination of the extent of the encapsulation based on the comparison. For example, knowing that fibrous collagen gradually increases around a stimulation lead, impedance values of tissue surrounding a stimulation lead can be measured at these frequencies and at various times in a controlled setting. These reference impedance values, in association with each stage of the encapsulation process, can then be stored in the SCS system 10. Subsequently, the impedances at each electrode 26 (or alternatively, each of a subset of the electrodes 26, which may include only one electrode 26) can be measured at all of the frequencies. For all of the frequencies, these newly acquired impedance values for each electrode 26 can then be compared to the reference impedance values associated with each stage of the encapsulation process. The stage associated with the reference impedance values that best matches the newly acquired impedance values, will be determined to be the state of the current encapsulation process with respect to the stimulation leads 12. Once the state of the encapsulation process is determined, the SCS system 10 can perform a correction or other action, such as programming the electrodes 26 with new stimulation parameters (e.g., increase or decrease the amplitude value) in order to restore the original therapy and/or providing a notification message or warning to the patient, so that the patient will know not to perform rigorous exercises during the early stages of the encapsulation process or will feel free to perform rigorous exercises during at the last stage of the encapsulation process.

As still another example, if the function performed by the SCS system 10 includes determining a posture or activity level of the patient, the analysis may involve comparing the impedance values acquired at the respective frequencies for each electrode 26 to known impedance values at those frequencies as a function of the posture and/or activity level of the patient, and making a determination of the extent of the encapsulation based on the comparison. For example, at the time of implantation, the impedance values of tissue surrounding the stimulation leads 12 can be measured at these frequencies as the patient assumes different postures (e.g., sitting, standing, laying down, etc.) and/or performs different physical activities (level of physical activity or type of physical activity, such as running, swimming, stretching) of the patient. These reference impedance values, in association with each posture and/or physical activity, can then be stored in the SCS system 10. Subsequently, the impedances at each electrode 26 (or alternatively, each of a subset of the electrodes 26, which may include only one electrode 26) can be measured at all of the frequencies. For all of the frequencies, these newly acquired impedance values for each electrode 26 can then be compared to the reference impedance values associated with each posture and/or physical activity of the patient. The posture and/or physical activity associated with the reference impedance values that best matches the newly acquired impedance values, will be determined to be the posture and/or physical activity of the patient. The SCS system 10 can perform a correction or other action, such as programming the electrodes 26 with new stimulation parameters if it is determined that the patient has been laying down or has very little physical activity indicating that the previous therapy was not effective.

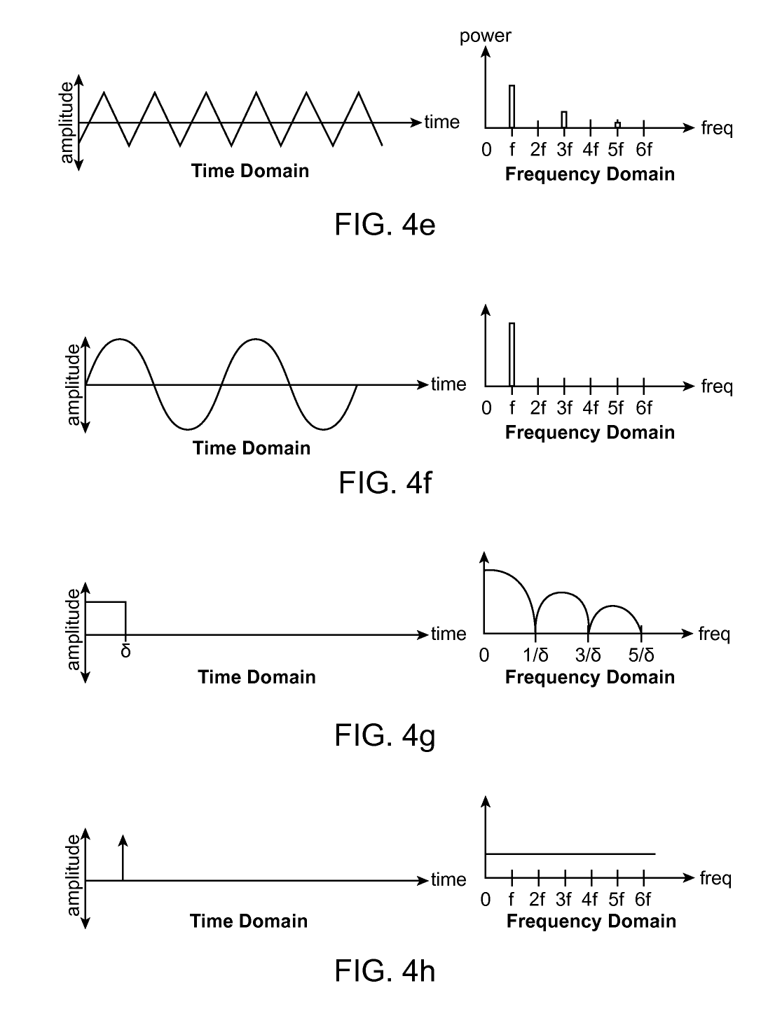

Turning now to FIG. 5, the main internal components of the IPG 14 will now be described. The IPG 14 includes analog output circuitry 50 configured for generating electrical energy in accordance with a defined therapeutic electrical pulse train having a specified pulse amplitude, pulse rate, pulse duration, pulse shape, and burst rate under control of control logic 52 over data bus 54. Control of the pulse rate and pulse duration of the electrical waveform is facilitated by timer logic circuitry 56, which may have a suitable resolution, e.g., 10 μs. The therapeutic electrical pulse train generated by the analog output circuitry 50 is output via capacitors C1-C16 to electrical terminals 58 corresponding to the electrodes 26. The therapeutic electrical pulse train can either be designed to be super-threshold (evoking paresthesia) or sub-threshold (no paresthesia).

For example, an exemplary super-threshold pulse train may be delivered at a relatively high pulse amplitude (e.g., 5 ma), a relatively low pulse rate (e.g., less than 1500 Hz, preferably less than 500 Hz), and a relatively high pulse width (e.g., greater than 100 μs, preferably greater than 200 μs). An exemplary sub-threshold pulse train may be delivered at a relatively low pulse amplitude (e.g., 2.5 ma), a relatively high pulse rate (e.g., greater than 1500 Hz, preferably greater than 2500 Hz), and a relatively low pulse width (e.g., less than 100 μs, preferably less than 50 μs).

As will be described in further detail below, the analog output circuitry 50 may also generate electrical energy designed to measure tissue impedance. Such electrical energy can take the form of an electrical pulse train, a continuous waveform (e.g., a sinusoidal waveform), or even a single pulse or impulse. Examples of electrical energy waveforms that can be used to measure tissue impedance are illustrated in FIGS. 4a-4h. Notwithstanding the nature and function of the delivered electrical energy, the analog output circuitry 50 may either comprise independently controlled current sources for providing modulation pulses of a specified and known amperage to or from the electrodes 26, and/or independently controlled voltage sources for providing modulation pulses of a specified and known voltage at the electrodes 26.

Any of the N electrodes may be assigned to up to k possible groups or timing “channels.” In one embodiment, k may equal four. The timing channel identifies which electrodes are selected to synchronously source or sink current to create an electric field in the tissue to be stimulated. Thus, multiple timing channels can be utilized to concurrently deliver electrical current (by interlacing the pulses of electrical pulse trains together) to multiple tissue regions of the patient. Amplitudes and polarities of electrodes on a channel may vary, e.g., as controlled by the RC 16. External programming software in the CP 18 is typically used to set modulation parameters including amplitude, pulse rate and pulse duration for the electrodes of a given channel, among other possible programmable features.

The N programmable electrodes can be programmed to have a positive (sourcing current), negative (sinking current), or off (no current) polarity in any of the k channels. Moreover, each of the N electrodes can operate in a multipolar (e.g., bipolar) mode, e.g., where two or more electrode contacts are grouped to source/sink current at the same time. Alternatively, each of the N electrodes can operate in a monopolar mode where, e.g., the electrodes associated with a channel are configured as cathodes (negative), and the case electrode (i.e., the IPG case) is configured as an anode (positive).

Further, the amplitude of the current pulse being sourced or sunk to or from a given electrode may be programmed to one of several discrete current levels, e.g., between 0 to 10 mA in steps of 0.1 mA. Also, the pulse duration of the current pulses is preferably adjustable in convenient increments, e.g., from 0 to 1 milliseconds (ms) in increments of 10 microseconds (μs). Similarly, the pulse rate is preferably adjustable within acceptable limits, e.g., from 0 to 50 k pulses per second (pps). Other programmable features can include slow start/end ramping, burst modulation cycling (on for X time, off for Y time), interphase, and open or closed loop sensing modes.

The operation of this analog output circuitry 50, including alternative embodiments of suitable output circuitry for performing the same function of generating modulation pulses of a prescribed amplitude and duration, is described more fully in U.S. Pat. Nos. 6,516,227 and 6,993,384, which are expressly incorporated herein by reference.

The IPG 14 further comprises monitoring circuitry 60 for monitoring the status of various nodes or other points 62 throughout the IPG 14, e.g., power supply voltages, temperature, battery voltage, and the like. Notably, the electrodes 26 fit snugly within the epidural space of the spinal column, and because the tissue is conductive, electrical measurements can be taken from the electrodes 26. Significantly, as discussed above, the monitoring circuitry 60 is configured for taking such electrical measurements at various frequencies in the manner described above. In the illustrated embodiment, the electrical measurements taken by the monitoring circuitry 60 are electrical impedance measurements. Electrical data can be measured using any one of a variety means. For example, the electrical data measurements can be made on a sampled basis during a portion of the time while a therapeutic modulation pulse is being applied to the tissue, or immediately subsequent to a therapeutic modulation pulse, as described in U.S. Pat. No. 7,742,823, which has previously been incorporated herein by reference. Alternatively, the electrical data measurements can be made independently of the electrical stimulation pulses, such as described in U.S. Pat. Nos. 6,516,227 and 6,993,384, which are expressly incorporated herein by reference.

To facilitate measurement of the tissue impedance, electrical signals can be transmitted between electrodes carried by one of the neurostimulation lead 12 and one or more other electrodes (e.g., electrodes on the same neurostimulation lead 12, electrodes on the other neurostimulation lead 12, the case 40 of the IPG 12, or an electrode affixed to the target tissue), and then electrical parameters can be measured in response to the transmission of the electrical signals.

For example, a known current (in the case where the IPG 14 is sourcing current) can be applied between a pair of electrodes 26 (or the case electrode 40), a voltage between the electrodes 26 can be measured, and an impedance between the electrodes 26 can be calculated as a ratio of the measured voltage to known current. Or a known voltage (in the case where the IPG is sourcing voltage) can be applied between a pair of electrodes 26, a current between the electrodes 26 can be measured, and an impedance between the electrodes 26 can be calculated as a ratio of the known voltage to measured current. As discussed above, the impedance can be measured in the time-domain, the frequency domain, or as related to other basis functions. If several impedance values are measured from a single electrical signal, the monitoring circuitry can include a single decomposition analyzer for measuring the impedance values using any specific basis function or basis functions, and in this case, may use a frequency spectrum analyzer for measuring the impedance values at frequencies corresponding to different sinusoidal frequency components in the electrical signal.

As another example, a field potential measurement technique may be performed by generating an electrical field at selected ones of the electrodes 26 and recording the electrical field at other selected ones of the lead electrodes 26. This may be accomplished in one of a variety of manners. For example, an electrical field may be generated conveying electrical energy to a selected one of the electrodes 26 and returning the electrical energy at the IPG case. Alternatively, multipolar configurations (e.g., bipolar or tripolar) may be created between the lead electrodes 26. Or, an electrode that is sutured (or otherwise permanently or temporarily attached (e.g., an adhesive or gel-based electrode) anywhere on the patient's body may be used in place of IPG outer case or lead electrodes 26. In either case, while a selected one of the electrodes 26 is activated to generate the electrical field, a selected one of the electrodes 26 (different from the activated electrode) is operated to record the voltage potential of the electrical field.

The IPG 14 further comprises a processor/controller in the form of a microcontroller (μC) 64 that controls the control logic over data bus 66, and obtains status data from the monitoring circuitry 60 via data bus 68. The IPG 14 additionally controls the timer logic 58. The IPG 14 further comprises memory 70 and oscillator and clock circuitry 72 coupled to the microcontroller 64. The microcontroller 64, in combination with the memory 70 and oscillator and clock circuitry 72, thus comprise a microprocessor system that carries out a program function in accordance with a suitable program stored in the memory 70. Alternatively, for some applications, the function provided by the microprocessor system may be carried out by a suitable state machine.

Thus, the microcontroller 64 generates the necessary control and status signals, which allow the microcontroller 64 to control the operation of the IPG 14 in accordance with a selected operating program and stimulation program stored in the memory 70. In controlling the operation of the IPG 14, the microcontroller 64 is able to individually generate an electrical pulse train at the electrodes 26 using the analog output circuitry 50, in combination with the control logic 52 and timer logic 56, thereby allowing each electrode 26 to be paired or grouped with other electrodes 26, including the monopolar case electrode. In accordance with stimulation parameters stored within the memory 70, the microcontroller 64 may control the polarity, amplitude, rate, pulse duration and timing channel through which the modulation pulses are provided. The microcontroller 64 is also able to generate a suitable electrical signal at the electrodes 26 using the analog output circuitry 50, and measuring the electrical impedance, or alternatively the field potential, using the monitoring circuitry 60. In the illustrated embodiment, the microcontroller 64 is capable of analyzing the impedance values and performing any necessary function (e.g., reprogramming the electrodes 26 or notifying the user via the RC 16 or CP 18) based on this analysis, as discussed above. To this end, the memory 70 stores any reference or threshold impedance values to which the measured impedance values can be compared during the analysis.

The IPG 14 further comprises an alternating current (AC) receiving coil 74 for receiving programming data (e.g., the operating program, modulation programs including the parameters, and/or a time schedule) from the RC 16 (shown in FIG. 1) in an appropriate modulated carrier signal, and charging and forward telemetry circuitry 76 for demodulating the carrier signal it receives through the AC receiving coil 74 to recover the programming data, which programming data is then stored within the memory 70, or within other memory elements (not shown) distributed throughout the IPG 14.

The IPG 14 further comprises back telemetry circuitry 78 and an alternating current (AC) transmission coil 80 for sending informational data sensed through the monitoring circuitry 60 to the RC 16. The back telemetry features of the IPG 14 also allow its status to be checked. For example, when the RC 16 initiates a programming session with the IPG 14, the capacity of the battery is telemetered, so that the external programmer can calculate the estimated time to recharge. Any changes made to the current modulation parameters are confirmed through back telemetry, thereby assuring that such changes have been correctly received and implemented within the implant system. Moreover, upon interrogation by the RC 16, all programmable settings stored within the IPG 14 may be uploaded to the RC 16. Significantly, the back telemetry features allow measured impedance values (if required to be processed by the RC 16 or CP 18) and any data related to prompting the RC 16 or CP 18 to generate notification messages or warnings to the patient to be transmitted to the RC 16 or CP 18.

The IPG 14 further comprises a rechargeable power source 82 and power circuitry 84 for providing the operating power to the IPG 14. The rechargeable power source 82 may, e.g., comprise a lithium-ion or lithium-ion polymer battery. The rechargeable battery 82 provides an unregulated voltage to the power circuitry 84. The power circuitry 84, in turn, generate the various voltages 86, some of which are regulated and some of which are not, as needed by the various circuits located within the IPG 14. The rechargeable power source 82 is recharged using rectified AC power (or DC power converted from AC power through other means, e.g., efficient AC-to-DC converter circuits, also known as “inverter circuits”) received by the AC receiving coil 134. To recharge the power source 82, an external charger (not shown), which generates the AC magnetic field, is placed against, or otherwise adjacent, to the patient's skin over the implanted IPG 14. The AC magnetic field emitted by the external charger induces AC currents in the AC receiving coil 74. The charging and forward telemetry circuitry 76 rectifies the AC current to produce DC current, which is used to charge the power source 82. While the AC receiving coil 74 is described as being used for both wirelessly receiving communications (e.g., programming and control data) and charging energy from the external device, it should be appreciated that the AC receiving coil 74 can be arranged as a dedicated charging coil, while another coil, such as coil 80, can be used for bi-directional telemetry.

It should be noted that the diagram of FIG. 5 is functional only, and is not intended to be limiting. Those of skill in the art, given the descriptions presented herein, should be able to readily fashion numerous types of IPG circuits, or equivalent circuits, that carry out the functions indicated and described, which functions include not only producing a stimulus current or voltage on selected groups of electrodes, but also the ability to measure electrical parameter data at an activated or non-activated electrode.

Additional details concerning the above-described and other IPGs may be found in U.S. Pat. No. 6,516,227, U.S. Patent Publication No. 2003/0139781, and U.S. patent application Ser. No. 11/138,632, entitled “Low Power Loss Current Digital-to-Analog Converter Used in an Implantable Pulse Generator,” which are expressly incorporated herein by reference. It should be noted that rather than an IPG, the SCS system 10 may alternatively utilize an implantable receiver-modulator (not shown) connected to the modulation leads 12. In this case, the power source, e.g., a battery, for powering the implanted receiver, as well as control circuitry to command the receiver-modulator, will be contained in an external controller inductively coupled to the receiver-modulator via an electromagnetic link. Data/power signals are transcutaneously coupled from a cable-connected transmission coil placed over the implanted receiver-modulator. The implanted receiver-modulator receives the signal and generates the modulation in accordance with the control signals.

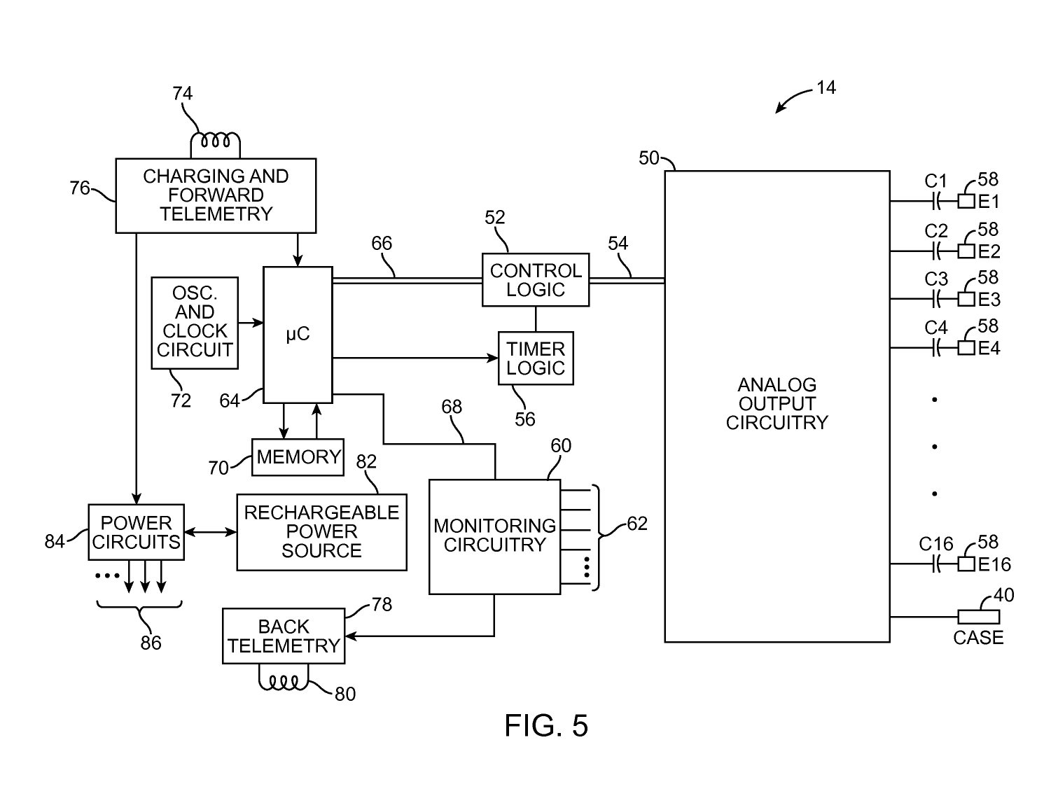

Referring now to FIG. 6, one exemplary embodiment of an RC 16 will now be described. As previously discussed, the RC 16 is capable of communicating with the IPG 14, CP 18, or ETS 20. The RC 16 comprises a casing 100, which houses internal componentry (including a printed circuit board (PCB)), a lighted display screen 102, an audio transducer (speaker) 103, and a control pad 104 carried by the exterior of the casing 100. In the illustrated embodiment, the display screen 102 is a lighted flat panel display screen, and the control pad 104 comprises a membrane switch with metal domes positioned over a flex circuit, and a keypad connector connected directly to a PCB. In an optional embodiment, the display screen 102 has touchscreen capabilities. The speaker 103 and/or display screen 102 may be used to provide notification messages or warnings to the user (e.g., if the stimulation leads 12 have migrated, the state of the encapsulation process, etc.). The control pad 104 includes a multitude of buttons 106, 108, 110, and 112, which allow the IPG 14 to be turned ON and OFF, provide for the adjustment or setting of stimulation parameters within the IPG 14, and provide for selection between screens.

In the illustrated embodiment, the button 106 serves as an ON/OFF button that can be actuated to turn the IPG 14 ON and OFF. The button 108 serves as a select button that allows the RC 16 to switch between screen displays and/or parameters. The buttons 110 and 112 serve as up/down buttons that can be actuated to increase or decrease any of stimulation parameters of the pulse generated by the IPG 14, including pulse amplitude, pulse width, and pulse rate.

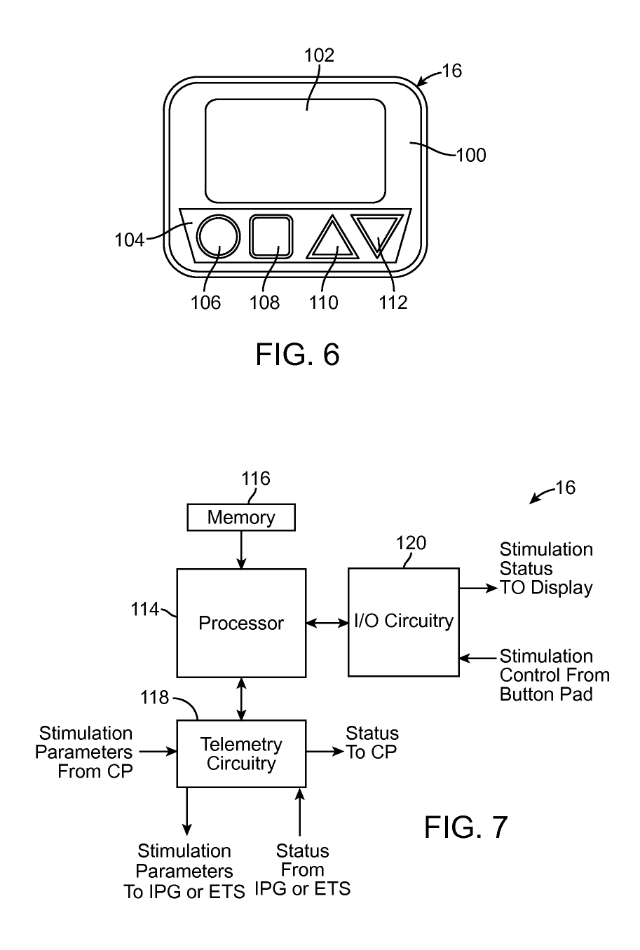

Referring to FIG. 7, the internal components of an exemplary RC 16 will now be described. The RC 16 generally includes a controller/processor 114 (e.g., a microcontroller), memory 116 that stores an operating program for execution by the controller/processor 114, and telemetry circuitry 118 for transmitting control data (including stimulation parameters and requests to provide status information) to the IPG 14 and receiving status information (including the measured electrical data) from the IPG 14 via link 34 (or link 32) (shown in FIG. 1), as well as receiving the control data from the CP 18 and transmitting the status data to the CP 18 via link 36 (shown in FIG. 1). Although the multi-frequency impedance analysis technique has been described as being performed by the microcontroller 62 of the IPG 14, it should be appreciated that this technique may be performed by the controller/processor 114 of the RC 16 (or a controller/processor in the CP 18). In this case, the RC 16 (or CP 18) may either perform the corrective action or instruct the IPG 14 to perform the corrective action. The RC 16 further includes input/output circuitry 120 for receiving stimulation control signals from the control pad 104 and transmitting operational status information to the display screen 102 and speaker 103 (shown in FIG. 6). Notably, while the controller/processor 80 is shown in FIG. 7 as a single device, the processing functions and controlling functions can be performed by a separate controller and processor. Thus, it can be appreciated that the controlling functions described below as being performed by the RC 16 can be performed by a controller, and the processing functions described below as being performed by the RC 16 can be performed by a processor. Further details of the functionality and internal componentry of the RC 16 are disclosed in U.S. Pat. No. 6,895,280, which has previously been incorporated herein by reference.

Although particular embodiments of the present inventions have been shown and described, it will be understood that it is not intended to limit the present inventions to the preferred embodiments, and it will be obvious to those skilled in the art that various changes and modifications may be made without departing from the spirit and scope of the present inventions. Thus, the present inventions are intended to cover alternatives, modifications, and equivalents, which may be included within the spirit and scope of the present inventions as defined by the claims.

What is claimed is:

1. A medical system configured for performing a medical function in a patient, comprising:
a medical lead configured for being implanted adjacent a tissue region of the patient;
an electrode configured for being implanted adjacent the tissue region;
analog output circuitry configured for delivering one or more electrical signals having a plurality of different sinusoidal frequency components to the tissue region via the electrode;
monitoring circuitry configured for measuring a plurality of different frequency-dependent electrical parameter values in response to the delivery of the one or more electrical signals to the tissue region; and
at least one controller/processor configured for analyzing the different electrical parameter values, and performing a function based on the different analyzed electrical parameter values.

2. The medical system of claim 1, wherein the electrode is carried by the medical lead.

3. The medical system of claim 1, wherein the monitoring circuitry is configured for measuring the different parameter values at the electrode.

4. The medical system of claim 1, wherein the electrical parameter values are impedance values.

5. The medical system of claim 1, wherein the one or more electrical signals comprises at least one monopolar electrical signal and at least one multipole electrical signal, the electrical parameter values comprise a plurality of monopolar impedance values and a plurality of multipolar impedance values, and the controller/processor is configured for analyzing the electrical parameter values by computing differences between the monopolar impedance values and the multipolar impedance values.

6. The medical system of claim 1, wherein the electrical parameter values are one or both of a resistance and a reactance.

7. The medical system of claim 1, wherein the one or more electrical signals comprises a single electrical signal, and the monitoring circuitry includes a signal decomposition analyzer configured for measuring the electrical parameter values using any specific basis function or functions.

8. The medical system of claim 7, wherein the signal decomposition analyzer is a frequency spectrum analyzer configured for measuring the electrical parameter values at frequencies corresponding to the different sinusoidal frequency components.

9. The medical system of claim 7, wherein the single electrical signal comprises an electrical pulse train.

10. The medical system of claim 1, wherein the one or more electrical signals comprises a plurality of different electrical signals having different frequency component profiles, and the monitoring circuitry is configured for measuring the electrical parameter values respectively in response to the delivery of the different electrical signals to the tissue region.

11. The medical system of claim 10, wherein the plurality of electrical signals comprises a plurality of electrical pulse trains having different pulse rates and/or different pulse widths.

12. The medical system of claim 10, wherein the plurality of electrical signals comprises a plurality of sinusoidal signals having different frequencies.

13. The medical system of claim 1, wherein the function is determining a migration of the medical lead.

14. The medical system of claim 1, wherein the function is determining a state of an encapsulation process with respect to the medical lead.

15. The medical system of claim 1, wherein the function is determining a posture and/or physical activity of the patient.

16. The medical system of claim 1, wherein the function is a corrective action.

17. The medical system of claim 16, wherein the medical lead carries at least one electrode, the analog output circuitry is configured for delivering therapeutic electrical energy to the electrode, the at least one controller/processor is configured for programming the at least one electrode with a set of neuromodulation parameters prior to performing the function, and the corrective action is modifying the neuromodulation parameter set and reprogramming the at least one electrode with the modified neuromodulation parameter set.

18. The medical system of claim 16, wherein the corrective action is providing a notification message or warning to a user.

19. The medical system of claim 1, further comprising memory storing at least one reference value, wherein the at least one controller/processor is configured for analyzing the electrical parameter values by comparing the electrical parameter values to the at least one reference value, and performing the function based on the comparison.

20. The medical system of claim 1, further comprising an implantable casing containing the analog output circuitry, monitoring circuitry, and the controller/processor.

21. The medical system of claim 1, further comprising an external control device containing the at least one controller/processor.
